(19) United States
(12) Patent Application Publication
(10) Pub. No.: US 2024/0058500 A1
(43) Pub. Date: Feb. 22, 2024

(54) DEVICE FOR DISINFECTING A FLUID FLOW IN A CONDUIT BY MEANS OF UV-C RADIATION

(71) Applicant: ISTITUTO NAZIONALE DI ASTROFISICA, ROMA (IT)

(72) Inventors: Matteo LOMBINI, ROMA (IT); Andrea BIANCO, ROMA (IT); Fausto CORTECCHIA, ROMA (IT); Emiliano DIOLAITI, ROMA (IT); Luigi LESSIO, ROMA (IT); Giuseppe MALAGUTI, ROMA (IT); Giovanni PARESCHI, ROMA (IT); Alessio ZANUTTA, ROMA (IT)

(21) Appl. No.: 18/267,356

(22) PCT Filed: Dec. 15, 2021
(86) PCT No.: PCT/IB2021/061779

§ 371 Date: Jun. 14, 2023

(30) Foreign Application Priority Data
Dec. 15, 2020 (IT) 102020000030899

(51) Int. Cl. A61L 9/18; C02F 1/32

(52) U.S. Cl. CPC A61L 9/18; C02F 1/325; A61L 2209/12; C02F 2201/3228; C02F 2201/3222; C02F 2303/04

(57) ABSTRACT

Device for disinfecting a fluid in a conduit comprising an outer casing adapted to be connected to at least a portion of said conduit and having a longitudinal axis and a source of UV-C radiation housed in the casing, wherein the casing includes at least one side wall having a longitudinal section with a curved profile with the concavity facing the interior of the casing.

### CROSS-REFERENCE TO RELATED APPLICATIONS

This patent application claims priority from Italian patent application no. 102020000030899 filed on Dec. 15, 2020, the entire disclosure of which is incorporated herein by reference.

### TECHNICAL FIELD

The present invention relates to a device for disinfecting a fluid flow in a conduit by means of UV-C radiation. The invention is preferably applied for disinfecting air in conduits of air conditioning or ventilation systems in general, which will be hereinafter referred to without losing generality. An alternative application is for disinfecting water or other fluids.

### BACKGROUND ART

In this emergency period related to the Covid-19 pandemic, controlling the virus spreading in the air is one of the most important tools for reducing disease transmission.

It has been shown that the virus can spread through the air, typically in aerosols generated from coughing, sneezing and breathing by infected people, over long distances and for considerable periods of time.

In particular, cases of virus transmission through ventilation conduits have been proven; this constitutes a non-negligible risk factor in all public places (offices, shops, department stores, means of mass transport) or private environments subject to air recirculation.

The disinfecting power of UV radiation is well known; however, UV disinfection systems known to be effective, particularly with wavelengths operating in the so-called UV-C band (ranging from 250 to 280 nm), require a dosage of several mJ/cm² (varying depending on the micro-organism considered) that can only be achieved by using relatively high-power sources and/or a relatively long residence time of the air in a confined area subject to radiation.

It follows that the known systems have considerable application limits. For example, they are not adapted to disinfect high flow-rate flows such as those within ventilation conduits.

### DISCLOSURE OF INVENTION

Aim of the present invention is to make a device for disinfecting a fluid flow which is free from the drawbacks of the known and above-specified devices.

The aforesaid aim is achieved by a device according to claim 1.

Thanks to the curved profile of the device walls, radiation is concentrated in the device itself, thus enhancing the sterilising action. This makes it possible to sterilise larger flow rates of fluid.

### BRIEF DESCRIPTION OF THE DRAWINGS

For a better understanding of the present invention, some preferred embodiments are described below with reference to the accompanying drawings, wherein:

FIG. 1 is a schematic median section of a disinfection device according to a first embodiment of the invention;

FIG. 2 is a section of the device along line II-II in FIG. 1;

FIG. 3 is a schematic section of a second embodiment of the invention; and

FIG. 4 is a section of a device according to the comparative examples.

### BEST MODE FOR CARRYING OUT THE INVENTION

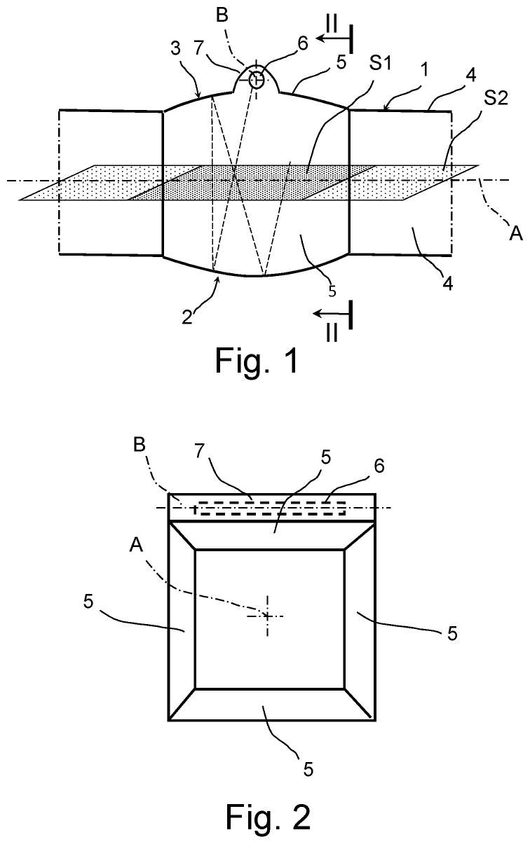

With reference to FIG. 1, number 1 denotes a ventilation conduit of axis A comprising a disinfecting device 2 according to the present invention.

The conduit 1 has a constant cross-section throughout its length, e.g. 300×300 mm square, apart from an intermediate tract 3 constituting a casing for the disinfection device 2.

The casing 3 has walls 5 with a curved profile, connected with the plane walls 4 of the conduit 1 to the respective axial ends; in particular, each wall 5, seen in section with a longitudinal plane perpendicular to the walls 5 connected thereto, has a curved profile with the concavity facing the interior of the casing 3.

The casing 3 therefore has a “convex” shape with a square cross-section varying from a minimum section at the ends, coinciding with the section of the conduit 1, to a maximum section at a longitudinal centreline of the casing.

In the example shown, the device 2 has an axial length of 0.5 m, and the radius of curvature of the walls 5 with a curved profile is constant and equal to 1 m; the walls 5 are therefore made up of portions of a cylinder with an axis perpendicular to the axis A.

The conduit 1 (partially shown) has an overall length of 5.5 m (including the device 2).

The device 2 comprises a source 6 of UV-C radiation which, in the example shown, consists of a mercury vapour discharge lamp.

The source 6 has an elongated cylindrical shape, of axis B, in the example shown having a length of 240 mm and a radius of 9 mm.

### Example 1 (FIGS. 1 and 2)

The source 6 is arranged with the axis B transverse to the conduit 1 inside a parabolic reflector 7 defining a transverse recess along a centreline of a wall 4.

The reflector 7 has a maximum width of 100 mm and a depth of 50 mm, with a minimum radius of curvature of 25 mm at the apex of the parabola. The section of maximum amplitude, wherein the reflector joins the wall 5, is placed at 180 mm from the axis A.

The axis of the source 6 is located in the focus of the reflector 7 at a distance of approximately 395 mm from the opposite wall 5.

The inner surfaces of the reflector 7, walls 4 and walls 5 are reflective.

In this disclosure and in the claims, the term “reflective” means a surface having a reflectivity greater than 80%.

In this specific example, the reflectivity considered is equal to 93%. An example of a usable material is Miro® UVC from Alanod GmbH & Co. KG.

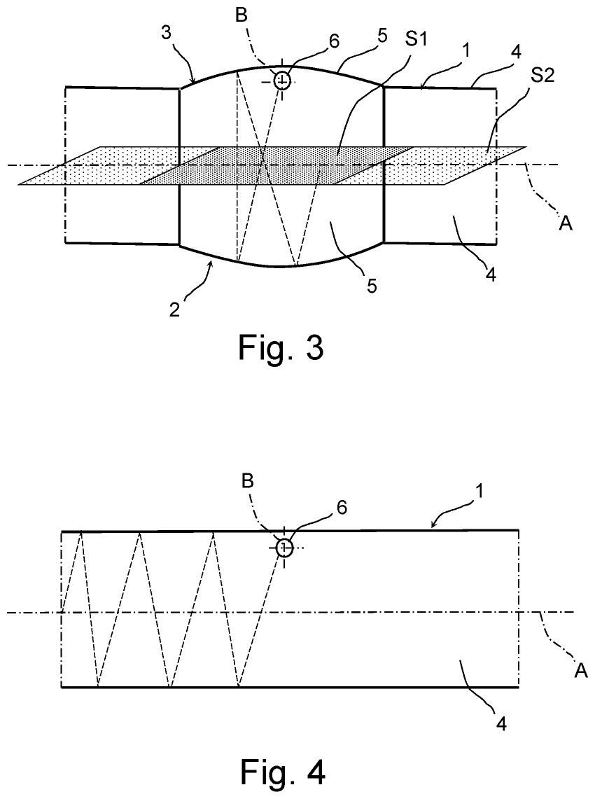

### Example 2 (FIG. 3)

The example 2 differs from the example 1 in that the source 6 is arranged inside the casing 3, along a centreline of a wall 5 of the device 2.

### Comparative Example 1 (FIG. 4)

The comparative example 1 differs from examples 1 and 2 in that the walls 5 of the device 2 are not curved. Therefore, the conduit 1 has a constant square cross-section.

The source 6 is arranged with the axis B transverse with respect to the conduit 1 in proximity of a wall 4 of conduit 1.

The reflectivity of the inner walls of the conduit 1 is as in example 1.

### Comparative Example 2 (FIG. 4)

The comparative example 2 is the same as comparative example 1, but the reflectivity of the inner surfaces of conduit 1 is low (5%).

Comparison

In order to evaluate the performance of the disinfection device 2 according to the various examples, the irradiation was calculated in two detection rectangular longitudinal median sections S1 and S2 (FIGS. 1, 3), placed along the axis A and parallel to the axis B of the source 6, having a width equal to the width of the conduit 1 (300 mm) and extending in length respectively for the length of the single device 2 (0.5 m) and for the entire length of the conduit 1 (5.5 m).

For ease of comparison, a unit power (1 W) of the source was considered.

For simplicity, it was assumed that the flow in the conduit is laminar with a constant velocity, and that the velocity remains unchanged inside the device 2 despite the increase in area. This is a basically plausible and certainly conservative hypothesis.

The total radiation power on sections S1 and S2 was then detected for each example.

Finally, the average power on sections S1 and S2 was calculated for each example by dividing the total power by the area of the respective sections.

The results are summarised in the table below:

| | | | | | Comparative examples | | | |
|---|---|---|---|---|---|---|---|---|
| | Example 1 | | Example 2 | | 1 | | 2 | |
| Total power S1 | 4990 | mW | 2860 | mW | 1410 | mW | 225 | mW |
| Total power S2 | 5030 | mW | 3090 | mW | 1700 | mW | 271 | mW |
| Power density | 3.3 | mW/cm² | 1.9 | mW/cm² | 0.94 | mW/cm² | 0.15 | mW/cm² |
| S1 (average) | | | | | | | | |
| Power density | 0.30 | mW/cm² | 0.18 | mW/cm² | 0.1 | mW/cm² | 0.016 | mW/cm² |
| S2 (average) | | | | | | | | |

From an examination of the results, it is clear that the use of a device 2 with curved profile side walls (example 2) leads substantially to a doubling of the average power density inside the device, due essentially to the greater permanence of the light beams generated by the source 6 inside the device 2, compared to a conduit with a constant cross-section (comparative example 1). It must be noted in particular the path of a radiation beam in FIG. 3, wherein the multiple reflections on the walls 5 tend to keep the beam inside the device 2, compared to the beam in FIG. 4 which tends to disperse along the conduit 1.

It is worth noting that in the comparative example 1, the total power in the detection section S2 exceeds the one in section S1 by approximately 20% (i.e., more than 20% of the power emitted by the source 6 “exits” the device 2 and is dispersed in the rest of the conduit), whereas in example 2, the total power in the detection section S2 exceeds the one in section S1 by approximately 8%, denoting a greater power concentration in the device 2.

By comparing the comparative example 1 and comparative example 2, both of which are not part of the present invention, it is possible to evaluate the contribution of reflectivity, with the geometrical characteristics of the conduit 1 and the source 6 being equal.

Comparing example 1 with example 2, it can be seen that the arrangement of the source 2 outside the curved profile of the wall 4 and inside the reflector 7 allows the power in the detection section S1 to be increased by about 75%, and the difference between the total power on section S2 and that on section S1 to be reduced to less than 1%. With this solution, therefore, the power emitted by the source 6 is almost entirely concentrated in device 2.

In order to calculate the radiation [mJ/cm²], it is sufficient to multiply the above-set forth average power values by the residence time of the flow inside the device 2 and conduit 1 respectively (and by the power of the source 6, which has so far been considered as unitary).

Finally, it is clear that modifications and variations can be made to the described embodiments without departing from the scope of protection defined by the claims.

In particular, the conduit 1 and the device 2 may be circular rather than square in cross-section; thus, the device 1 may be delimited by a continuous toroidal wall rather than walls curved in one direction only.

The profile of the side wall(s) of the device 2, in longitudinal section, may be defined by a curve with variable, rather than constant, radius, for example a parabola.

The source 6 can be of different types and operate at different wavelengths as long as they are effective in sanitising micro-organisms. For example, it may consist of an excimer lamp (operating at 222 nm) or one or more substantially point sources, such as LEDs (operating at approximately 275 nm), or a curved profile tubular source.

There could also be more sources in case more irradiation is needed. The sources could each be in their own reflector or arranged in the same reflector

The device 2 may be used to sterilise water (or another liquid) in a conduit. In this case, the above-noted advantages are joined by the further advantage that a liquid slows down in the device 2, due to the convex shape of the walls 5, and thus increases the residence time within the device itself.

1. A device for disinfecting a fluid in a conduit comprising:
an outer casing adapted to be connected to at least a portion of said conduit and having a longitudinal axis; and
at least one source of UV-C radiation housed in the casing,
characterized in that the casing includes at least one side wall having a longitudinal section with a curved profile with the concavity facing the interior of the casing.

2. A device as claimed in claim 1, wherein the casing has a reflective inner surface.

3. A device as claimed in claim 1, wherein the casing has a square cross-section, and comprises four walls having said curved profile in longitudinal section.

4. A device as claimed in claim 1, wherein the casing has a circular cross-section, and comprises a toroidal side wall.

5. A device as claimed in claim 1, wherein the source is arranged in proximity to the at least one side wall of the casing.

6. A device as claimed in claim 1, wherein the source is disposed within a reflector defining a cavity open towards the inside of the casing.

7. A device as claimed in claim 6, wherein the reflector defines a recess of said at least one side wall.

8. A device as claimed in claim 1, wherein the source is a mercury vapor discharge lamp.

9. A device as claimed in claim 4, wherein the source is an excimer lamp or includes one or more LEDs.

10. A device as claimed in claim 1, wherein the source is an elongated cylindrical lamp having an axis transverse to the axis of the housing.
